(19) United States
(12) Patent Application Publication
(10) Pub. No.: US 2008/0152719 A1
(43) Pub. Date: Jun. 26, 2008

(54) Multiparticulate Pharmaceutical Form Comprising Pellets With a Matrix Which Influences the Delivery of a Modulatory Substance

(75) Inventors: Hans-Ulrich Petereit, Darmstadt (DE); Rosario Lizio, Rossdorf (DE); Hema Ravishankar, Mumbai (IN); Ashwini Samel, Andheri (IN)

Correspondence Address: OBLON, SPIVAK, MCCLELLAND MAIER & NEUSTADT, P.C., 1940 DUKE STREET, ALEXANDRIA, VA, 22314, US

(73) Assignee: ROEHM GMBH, Darmstadt (DE)

(21) Appl. No.: 11/815,677

(22) PCT Filed: Mar. 3, 2006
(86) PCT No.: PCT/EP06/01950

§ 371 Date: Aug. 7, 2007

(30) Foreign Application Priority Data
Mar. 29, 2005 (IN) 325/CHE/2005

(51) Int. Cl. A61K 9/16

(52) U.S. Cl. 424/492; 424/491; 424/496; 424/497; 424/489

(57) ABSTRACT

The invention relates to a multiparticulate pharmaceutical form, comprising pellets with a multilayer structure for controlled active ingredient release, comprising a) optionally a neutral core (nonpareilles), b) an inner controlling layer comprising a substance having a modulating effect, which is embedded in a matrix which influences the delivery of the modulatory substance and which comprises pharmaceutically usable polymers, waxes, resins and/or proteins, and where appropriate an active ingredient, c) an active ingredient layer comprising an active pharmaceutical ingredient and, where appropriate, a substance having a modulating effect, d) an outer controlling layer comprising at least 60% by weight of one or a mixture of a plurality of (meth) acrylate copolymers where the outer controlling layer has a thickness from 20 to less than 55 μm and contains 0,1 to 10% by weight of glycerol monostearate, where the multiparticulate pharmaceutical form contains 20 to 60% by weight of the pellets, which are compressed in mixture with 80 to 40% by weight of an outer phase which consists from 50 to 100% by weight of a cellulose or a derivate of cellulose and optionally 0 to 50% by weight of further pharmaceutical excipients.

The invention relates to a multiparticulate pharmaceutical form comprising pellets with a matrix which influences the delivery of a modulatory substance.

### PRIOR ART

EP-A 0 463 877 describes pharmaceutical compositions with delayed active ingredient release consisting of a core with an active pharmaceutical ingredient as a monolayer coating film which comprises a water-repellent salt and a water-insoluble copolymer of ethyl acrylate, methyl methacrylate and trimethylammonium-ethyl methacrylate chloride. The water-repellent salt may be for example Ca stearate or Mg stearate. Sigmoidal release plots are obtained.

EP-A 0 225 085, EP-A 0 122 077 and EP-A 0 123 470 describe the use of organic acid in medicament cores which are provided with various coatings from organic solutions. Essentially sigmoidal release characteristics result.

EP-A 0 436 370 describes pharmaceutical compositions with delayed active ingredient release consisting of a core with an active pharmaceutical ingredient and an organic acid and an outer coating film which has been applied by aqueous spraying and is a copolymer of ethyl acrylate, methyl methacrylate and trimethylammonium-ethyl methacrylate chloride. In this case, sigmoidal release plots are likewise obtained.

WO 00/19984 describes a pharmaceutical preparation consisting of (a) a core comprising an active ingredient, where appropriate a carrier and

U.S. Pat. No. 5,508,040 describes a multiparticulate pharmaceutical form consisting of large number of pellets which are held together in a binder. The pellets have an active ingredient and an osmotically active modulator, e.g. NaCl or an organic acid, in the core. The pellet cores are provided with coatings of different thicknesses, e.g. composed of (meth)acrylate copolymers with quaternary amino groups. To reduce the permeability, the coatings also comprise hydrophobic substances, e.g. fatty acids, in amounts of 25% by weight or above. The multiparticulate pharmaceutical form is released through a the contained active ingredient in a large number of pulses which corresponds to the number of pellet populations with coatings of different thicknesses.

EP 1 064 938 A1 describes a pharmaceutical form which has an active ingredient and a surface-active substance (surfactant) in the core. The core may additionally comprise an organic acid and is coated with (meth)acrylate copolymers with quaternary amino groups. “Pulsatile” release plots are obtained. Stepped release plots can be obtained by combining pellets with different coatings in one pharmaceutical form.

WO 01/13895 describes bimodal release systems for active ingredients having a sedative hypnotic effect. The release profiles are achieved by mixtures of different pellet populations.

WO 01/37815 describes multilayer release systems for controlled, pulsatile delivery of active ingredients. In this case, an inner membrane which can be dissolved by the active ingredient formulation present in the cores is present. Also present is an outer membrane which additionally has a pore-forming substance.

WO 01/58433 describes multilayer release systems for controlled, pulsatile delivery of active ingredients. In this case, the active ingredient is present in the core and is surrounded by a polymer membrane which is soluble in intestinal juice. An outer membrane consists of a mixture of a polymer which is soluble in intestinal juice with a water-insoluble polymer in defined ranges of amounts. An intermediate layer comprising an organic acid may be present between the inner and outer membrane.

U.S. Pat. No. 5,292,522 refers to an aqueous film coating agent for solid medicaments. A water soluble lipophilic emulsifier having a hydrophile-lipophile balance (HLB) of 3.5 to 7 is added as a lubricant and parting agent to a polymer dispersion containing methacrylic type polymers in order to prevent resulting pharmaceutical dosage forms from sticking to one another.

WO 02/060415 A1 refers to a multiparticulate form of medicament, comprising at least two different coated forms of pellets. Gycerolmonosterate and talc are generally mentioned among other substances as parting agents. In the examples talc is used as a parting agent in the outer coating films of the pellets.

Problem and Solution

It was one object of the present invention to develop a multiparticulate pharmaceutical form which releases at least 50% of an active pharmaceutical ingredient in less than 8 hours in order to achieve acceptable drug absorption in vivo. Other object of the invention was that starting from EP-A 0 436 370 and WO 00/19984, it was intended to develop a pellet system for the multiparticulate pharmaceutical form that permits the permeability of film coatings to be influenced by intrinsic modulation so that release profiles with zero order, first order, first order with initial accelerated phase, slow-fast, fast-slow profiles can be adjusted individually depending on the active ingredient and therapeutic requirements.

The problem is solved by a

multiparticulate pharmaceutical form, comprising pellets with a multilayer structure for controlled active ingredient release, comprising
a) optionally a neutral core (nonpareilles),
b) an inner controlling layer comprising a substance having a modulating effect, which is embedded in a matrix which influences the delivery of the modulatory substance and which comprises pharmaceutically usable polymers, waxes, resins and/or proteins, and where appropriate an active ingredient,
c) an active ingredient layer comprising an active pharmaceutical ingredient and, where appropriate, a substance having a modulating effect,
d) an outer controlling layer comprising at least 60% by weight of one or a mixture of a plurality of (meth)acrylate copolymers composed of 98 to 85 C₁ to C₄ alkyl esters of (meth)acrylic acid and 2 to 15% by weight of methacrylate monomers with a quaternary amino group in the alkyl radical, and, where appropriate, up to 40% by weight of further pharmaceutically usable polymers,

where the layers may additionally and in a manner known per se comprise pharmaceutically usual excipients, where the outer controlling layer has a thickness from 20 to less than 55 μm and contains 0,1 to 10% by weight of glycerol monostearate, where the multiparticulate pharmaceutical form contains 20 to 60% by weight of the pellets, which are compressed in mixture with 80 to 40% by weight of an outer phase which consists from 50 to 100% by weight of a cellulose or a derivate of cellulose and optionally 0 to 50% by weight of further pharmaceutical excipients.

### IMPLEMENTATION OF THE INVENTION

The invention relates to a multiparticulate pharmaceutical form, comprising pellets with a multilayer structure for controlled active ingredient release comprising essentially an optional core a) and layers b), c) and d). It is also possible in addition for usual topcoat layers, which may for example be pigmented, to be present.

Optional Core a)

A neutral core (nonpareilles) may be present.

The Inner Controlling Layer b)

The inner controlling layer comprises a substance having a modulating effect, which is embedded in a matrix which influences the delivery of the modulatory substance and which comprises pharmaceutically usable polymers, waxes, resins and/or proteins or consists thereof, and additionally may comprise where appropriate an active ingredient. To assist the formulation it is possible to admix further pharmaceutically customary excipients such as, for example, binders such as cellulose and derivatives thereof, plasticizers, polyvinylpyrrolidone (PVP), humectants, disintegration promoters, lubricants, disintegrants, starch and derivatives thereof, sugars and/or solubilizers.

Suitable processes for producing the inner controlling layer b) are direct compression, compression of dry, wet or sintered granules, extrusion and subsequent rounding off, wet or dry granulation or direct pelleting (e.g. on plates) or, if an optional core a) is present, by binding powders (powder layering) onto active ingredient-free cores (nonpareilles).

The inner controlling layer b) influences the delivery of the substance having a modulating effect and of the active ingredient which is present where appropriate from the core layer. The inner controlling layer consists of pharmaceutically usable polymers, waxes, proteins and/or other pharmaceutically customary excipients.

Examples of suitable polymers are the following:

copolymers of methyl methacrylate and/or ethyl acrylate and methacrylic acid, copolymers of methyl methacrylate, methyl acrylate and methacrylic acid, copolymers of methyl methacrylate, butyl methacrylate and dimethylethyl methacrylate, copolymers of methyl methacrylate, ethyl acrylate and trimethylammoniumethyl methacrylate, copolymers of methyl methacrylate and ethyl acrylate, copolymers of ethyl acrylate, methyl acrylate, butyl methacrylate and methacrylic acid,

polyvinylpyrolidones (PVPs), polyvinyl alcohols, polyvinyl alcohol-polyethylene glycol graft copolymer (Kollicoat®), starch and derivatives thereof, polyvinyl acetate phthalate (PVAP, Coateric®), polyvinyl acetate (PVAC, Kollicoat), vinyl acetate/vinylpyrolidone copolymer (Kollidon® VA64), vinyl acetate: crotonic acid 9:1 copolymer (VAC: CRA, Kollicoat® VAC), polyethylene glycols with a molecular weight above 1000 (g/mol) and/or shellac,

celluloses such as, for example, anionic carboxymethylcellulose and salts thereof (CMC, Na-CMC, Ca-CMC, Blanose, Tylopur), carboxymethylethylcellulose (CMEC, Duodcell®), hydroxyethylcellulose (HEC, Klucel), hydroxypropylcellulose (HPC), hydroxypropylmethylcellulose (HPMC, Pharmacoat, Methocel, Sepifilm, Viscontran, Opadry), hydroxymethylethylcellulose (HEMC), ethylcellulose (EC, Ethocel®, Aquacoat®, Surelease®), methylcellulose (MC, Viscontran, Tylopur, Methocel), cellulose esters, cellulose glycolate, cellulose acetate phthalate (CAP, Cellulosi acetas, PhEur, cellulose acetate phthalate, NF, Aquateric®), cellulose acetate succinate (CAS), cellulose acetate trimeliate (CAT), hydroxypropylmethylcellulose phthalate (HPMCP, HP50, HP55), hydroxypropylmethylcellulose acetate succinate (HPMCAS-LF, -MF, -HF).

The inner controlling layer b) may preferably consist of a polymer or contain one which is insoluble in water or only swellable in water.

The inner controlling layer may consist of a wax such as, for example, carnauba wax and/or beeswax, or comprise the latter.

The inner controlling layer may comprise the resin shellac or consist thereof.

The inner controlling layer may comprise a protein such as, for example, albumin, gelatin, zein, gluten, collagen and/or lectins, or consist thereof. The protein of the inner controlling layer should preferably have no therapeutic function, as is the case with protein or peptide active ingredients, so that the technical effects of the active ingredient layer c) on the one hand and of the inner controlling layer b), if the latter comprises an active ingredient, on the other hand do not overlap where possible.

Substances having a Modulating Effect

Substances having a modulating effect which are to be used according to the invention may have a molecular weight of below 500, be in solid form and be ionic.

The substance having a modulating effect is preferably water-soluble.

The substance having a modulating effect may be for example an organic acid or the salt of an organic or inorganic acid.

The substance having a modulating effect may be for example succinic acid, citric acid, fumaric acid, malic acid, maleinic acid, tartaric acid, laurylsulphuric acid, a salt of these acids or a salt of the following anions: taurochlolate and other cholates, chlorides, acetates, lactates, phosphates and/or sulphates.

In the human and animal gastrointestinal tract the concentration of ions may vary to a certain extent and thus may influence the activity of the modulating substances. For reproducible in-vivo results substances having a modulating effect, which are not or only a little influenced by varying ionic strength are preferred. It was surprisingly found that sodium chloride, citric acid and sodium succinate have in-vitro almost the same activity in purified water and in phosphate buffer pH 6,8 (Pharm. Eur.). Therefore sodium chloride, citric acid and sodium succinate are the most preferred modulating substances in order to achieve reproducible in-vivo results.

Mode of Functioning of the Components with One Another

The mode of functioning of the substance having a modulating effect in the multilayer pharmaceutical form can be described approximately as follows: Na succinate (succinic acid), Na acetate and citric acid increase the rate of active ingredient delivery. NaCl and Na citrate decrease the rate of active ingredient delivery.

If the active ingredient layer c) comprises in addition to the inner core layer a) a substance having a modulating effect, the active ingredient delivery is determined firstly by the substance having a modulating effect which is present in the outer layer, the active ingredient layer c). If this substance is substantially consumed, the effect of the substance having a modulating effect in the inner layer, the inner controlling layer b), starts and determines further active ingredient release.

The various active ingredient delivery profiles can be adapted to the active ingredient and the therapeutic aim by combining different amounts of one and/or different substances having a modulating effect in the two layers. There is in addition the effect of the matrix itself which in turn itself controls delivery of the substance having a modulating effect.

The amount of active ingredient delivered is essentially controlled by the outer controlling layer d). If the inner controlling layer additionally comprises an active ingredient, this layer can be used to adjust the active ingredient delivery profile towards the end of active ingredient delivery.

If the active ingredients themselves comprise ionic groups or are present in the salt form, the active ingredient itself can influence the effect of the substance or substances having a modulating effect so that the latter is diminished or enhanced. This interaction can be utilized as further control element.

The Active Ingredient Layer c)

The active ingredient layer c) comprises an active pharmaceutical ingredient, and where appropriate a substance having a modulating effect, which may be identical to or different from the substance having a modulating effect of the core layer.

Active Ingredients

The multilayer pharmaceutical form of the invention is suitable in principle for any active ingredients. Medicinal substances in use can be found in reference works such as, for example, the Rote Liste or the Merck Index.

The active ingredients or medicinal substances employed for the purposes of the invention are intended to be used on or in the human or animal body in order
1. to cure, to alleviate, to prevent or to diagnose disorders, conditions, physical damage or pathological symptoms.
2. to reveal the condition, the status or the functions of the body or mental states.
3. to replace active substances or body fluids produced by the human or animal body.
4. to ward off, to eliminate or to render harmless pathogens, parasites or exogenous substances, or
5. to influence the condition, the status or the functions of the body or mental states.

These pharmaceutically active substances may belong to one or more active ingredient classes such as ACE inhibitors, adrenergics, adrenocorticosteroids, acne therapeutic agents, aldose reductase inhibitors, aldosterone antagonists, alpha-glucosidase inhibitors, alpha 1 antagonists, remedies for alcohol abuse, amino acids, amoebicides, anabolics, analeptics, anaesthetic additions, anaesthetics (non-inhalational), anaesthetics (local), analgesics, androgens, angina therapeutic agents, antagonists, antiallergics, antiallergics such as PDE inhibitors, antiallergics for asthma treatment, further antiallergics (e.g. leukotriene antagonists, antianaemics, antiandrogens, antianxiolytics, antiarthritics, antiarrhythmics, antiatheriosclerotics, antibiotics, anticholinergics, anticonvulsants, antidepressants, antidiabetics, antidiarrhoeals, antidiuretics, antidotes, antiemetics, antiepileptics, antifibrinolytics, antiepileptics, antihelmintics, antihistamines, antihypotensives, antihypertensives, antihypertensives, antihypotensives, anticoagulants, antimycotics, antiestrogens, antiestrogens (non-steroidal), antiparkinson agents, antiinflammatory agents, antiproliferative active ingredients, antiprotozoal active ingredients, antirheumatics, antischistosomicides, antispasmolytics, antithrombotics, antitussives, appetite suppressants, arteriosclerosis remedies, bacteriostatics, beta-blockers, beta-receptor blockers, bronchodilators, carbonic anhydrase inhibitors, chemotherapeutic agents, choleretics, cholinergics, cholinergic agonists, cholinesterase inhibitors, agents for the treatment of ulcerative colitis, cyclooxygenaze inhibitors diuretics, ectoparasiticides, emetics, enzymes, enzyme inhibitors, enzyme inhibitors, active ingredients to counter vomiting, fibrinolytics, fungistatics, gout remedies, glaucoma therapeutic agents, glucocorticoids, glucocorticosteroids, haemostatics, cardiac glycosides, histamine H2 antagonists, hormones and their inhibitors, immunotherapeutic agents, cardiotonics, coccidiostats, laxatives, lipid-lowering agents, gastrointestinal therapeutic agents, malaria therapeutic agents, migraine remedies, microbiocides, Crohn's disease, metastasis inhibitors, migraine remedies, mineral preparations, motility-increasing active ingredients, muscle relaxants, neuroleptics, active ingredients for treatment of estrogens, osteoporosis, otologicals, antiparkinson agents, phytopharmaceuticals, proton pump inhibitors, prostaglandins, active ingredients for treating benign prostate hyperblasia, active ingredients for treating pruritus, psoriasis active ingredients, psychoactive drugs, free-radical scavengers, renin antagonists, thyroid therapeutic agents, active ingredients for treating seborrhoea, active ingredients to counter seasickness, spasmolytics, alpha- and beta-sympathomimetics, platelet aggregation inhibitors, tranquilizers, ulcer therapeutic agents, further ulcer therapeutic agents, agents for the treatment of urolithiasis, virustatics, vitamins, cytokines, active ingredients for combination therapy with cytostatics, cytostatics.

Active Ingredients

Examples of suitable active ingredients are acarbose, acetylsalicylic acid, abacavir, aceclofenac, aclarubicin, acyclovir, actinomycin, adalimumab, adefovir, adefovirdipivoxil, adenosylmethionine, adrenaline and adrenaline derivatives, agalsidase alpha, agalsidase beta, alemtuzumab, almotriptan, alphacept, allopurinol, almotriptan, alosetron, alprostadil, amantadine, ambroxol, amisulpride, amlodipine, amoxicillin, 5-aminosalicylic acid, amitriptyline, amlodipine, amoxicillin, amprenavir, anakinra, anastrozole, androgen and androgen derivatives, apomorphine, aripiprazole, arsenic trioxide, artemether, atenolol, atorvastatin, atosiban, azathioprine, azelaic acid, barbituric acid derivatives, balsalazide, basiliximab, beclapermin, beclomethasone, bemiparin, benzodiazepines, betahistine, bexaroten, bezafibrate, bicalutamide, bimatoprost, bosentan, botulinus toxim, brimonidine, brinzolamide, budesonide, budipine, bufexamac, calcitonin, calcium antagonists, calcium salts, candesartan, capecitabine, captopril, carbamazepine, carifenacin, carvedilol, caspofungin, cefaclor, cefadroxil, cefalexin cefalosporins, cefditoren, cefprozil, celecoxib, cepecitabine, cerivastatim, cetirizine, cetrorelix, cetuximab, chenodeoxycholic acid, chorionic gonadotropin, ciclosporin, cidofovir, cimetidine, ciprofloxacin, cisplatin, cladribine, clarithromycin, clavulanic acid, clindamycin, clobutinol, clonidine, clopidogrel, codeine, caffeine, colestyramine, cromoglicic acid, cotrimoxazole, coumarin and coumarin derivatives, darbepoetin, cysteamine, cysteine, cytarabine, cyclophosphamide, cyproterone, cytarabine, daclizumab, dalfopristin, danaparoid, dapiprazole, darbepoetin, defepripone, desipramine, desirudin, desloaratadine, desmopressin, desogestrel, desonide, dexibuprofen, dexketoprofen, disoproxil, diazepam and diazepam derivatives, dihydralazine, diltiazem, dimenhydrinate, dimethyl sulphoxide, dimeticon, dipivoxil, dipyridarnoi, dolasetron, domperidone, and domperidane derivatives, donepzil, dopamine, doxazosin, doxorubizin, doxylamine, diclofenac, divalproex, dronabinol, drospirenone, drotrecogin alpha, dutasteride, ebastine, econazole, efavirenz, eletripan, emidastine, emtricitabine, enalapril, encepur, entacapone, enfurvirtide, ephedrine, epinephrine, eplerenone, epoetin and epoetin derivatives, eprosartan, eptifibatide, ertapenem, esomeprazole, estrogen and estrogen derivatives, etanercept, ethenzamide, ethinestradiol, etofenamate, etofibrate, etofylline, etonogestrel, etoposide, exemestan, exetimib, famciclovir, famotidine, faropenan daloxate, felodipine, fenofibrate, fentanyl, fenticonazole, fexofenadine, finasteride, fluconazole, fludarabine, flunarizine, fluorouracil, fluoxetine, flurbiprofen, flupirtine, flutamide, fluvastatin, follitropin, fomivirsen, fondaparinux, formoterol, fosfomicin, frovatriptan, furosemide, fusidic acid, gadobenate, galantamine, gallopamil, ganciclovir, ganirelix, gatifloxacin, gefitinib, gemfibrozil, gentamicin, gepirone, progestogen and progestogen derivatives, ginkgo, glatiramer, glibenclamide, glipizide, glucagon, glucitol and glucitol derivatives, glucosamine and glucosamine derivatives, glycoside antibiotics, glutathione, glycerol and glycerol derivatives, hypothalamus hormones, goserelin, grepafloxacin, gyrase inhibitors, guanethidine, gyrase inhibitors, haemin, halofantrine, haloperidol, urea derivatives as oral antidiabetics, heparin and heparin derivatives, cardiac glycosides, hyaluronic acid, hydralazine, hydrochlorothiazide and hydrochlorothiazide derivatives, hydroxyomeprazole, hydroxyzine, ibritumomab, ibuprofen, idarubicin, ifliximab, ifosfamide, iloprost, imatinib, imidapril, imiglucerase, imipramine, imiquimod, imidapril, indometacin, indoramine, infliximab, insulin, insulin glargin, interferons, irbesartan, irinotecan, isoconazole, isoprenaline, itraconazole, ivabradines, iodine and iodine derivatives, St. John's wort, potassium salts, ketoconazole, ketoprofen, ketotifen, lacidipine, lansoprazole, laronidase, latanoprost, leflunomide, lepirudin, lercanidipine, leteprinim, letrozole, levacetylmethadol, levetiracetam, levocetirizine, levodopa, levodrpropicin, levomethadone, licofelone, linezolide, lipinavir, lipoic acid and lipoic acid derivatives, lisinopril, lisuride, lofepramine, lodoxamide, lomefloxacin, lomustine, loperamide, lopinavir, loratadine, lornoxicam, losartan, lumefantrine, lutropine, magnesium salts, macrolide antibiotics, mangafodipir, maprotiline, mebendazole, mebeverine, meclozine, mefenamic acid, mefloquine, meloxicam, memantine, mepindolol, meprobamate, meropenem, mesalazine, mesuximide, metamizole, metformin, methadone, methotrexate, methyl 5-amino-4-oxopentanoate, methylnaloxone, methylnaloxone, methylnaltrexones, methylphenidate, methylprednisolone, metixen, metoclopramide, metoprolol, metronidazole, mianserin, mibefradil, miconazole, mifepristone, miglitol, miglustad, minocycline, minoxidil, misoprostol, mitomycin, mizolastine, modafinil, moexipril, montelukast, moroctocog, morphinans, morphine and morphine derivatives, moxifloxacin, ergot alkaloids, nalbuphine, naloxone, naproxen, naratriptan, narcotine, natamycin, nateglinide, nebivolol, nefazodone, nelfinavir, neostigmine, neramexan, nevirapine, nicergoline, nicethamide, nifedipine, niflumic acid, nimodipine, nimorazole, nimustine, nesiritide, nisoldipine, norfloxacin, novamine sulphone, noscapine, nystatin, ofloxacin, oktotride, olanzapine, olmesartan, olsalazine, oseltamivir, omeprazole, omoconazole, ondansetron, orlistat, oseltamivir, oxaceprol, oxacillin, oxaliplatin, oxaprozin, oxcarbacepin, oxicodone, oxiconazole, oxymetazoline, palivizumab, palanosetron, pantoprazole, paracetamol, parecoxib, paroxetine, pegaspargase, peginterferon, pegfilgrastrim, penciclovir, oral penicillins, pentazocine, pentifylline, pentoxifylline, peptide antibiotics, perindopril, perphenazine, pethidine, plant extracts, phenazone, pheniramine, phenylbutyric acid, phenytoin, phenothiazines, phenserine, phenylbutazone, phenytoin, pimecrolimus, pimozide, pindolol, pioglitazone, piperazine, piracetam, pirenzepine, piribedil, pirlindol, piroxicam, pramipexol, pramlintide, pravastatin, prazosin, procaine, promazine, propiverine, propranolol, propionic acid derivatives, propyphenazone, prostaglandins, protionamide, proxyphylline, quetiapine, quinapril, quinaprilate, quinupristine, ramipril, ranitidine, rabeprazole, raloxifen, ranolazine, rasburicase, reboxetin, repaclinides, reproterol, reserpine, revofloxacin, ribavirin, rifampicin, riluzoles, rimexolone, risedronate, risperidone, ritonavir, rituximab, rivastimen, risatriptan, rofecoxib, ropinirol, ropivacaine, rosiglitazone, roxatidine, roxithromycin, ruscogenin, rosuvastatin, rutoside and rutoside derivatives, sabadilla, salbutamol, salicylates, salmeterol, saperconazoles, thyroid hormones, scopolamine, selegiline, sertaconazole, sertindole, sertraline, sevelamer, sibutramine, sildenafil, silicates, simvastatin, sirolimus, sitosterol, sotalol, spaglumic acid, sparfloxacin, spectinomycin, spiramycin, spirapril, spironolactone, stavudine, streptomycin, sucralfate, sufentanil, sulbactam, sulphonamides, sulphasalazine, sulpiride, sultamicillin, sultiam, sumatriptan, suxamethonium chloride, tacrine, tacrolimus, tadalafil, taliolol, talsaclidine, tamoxifen, tasonermin, tazarotene, tegafur, tegaserod, telithromycin, telmisartan, temoporfin, temozolomide, tenatoprazole, tenecteplase, teniposide, tenofovir, tenoxicam, teriparatide, terazosin, terbinafine, terbutaline, terfenadine, teriparatide, terlipressin, tertatolol, testosterone and testosterone derivatives, tetracyclines, tetryzoline, tezosentan, theobromine, theophylline, theophylline derivatives, thiamazole, thiotepa, thr. growth factors, tiagabine, tiapride, tibolone, ticlopidine, tilidine, timolol, tinidazole, tioconazole, tioguanine, tiotropium, tioxolone, tirazetam, tiropramide, trofiban, tizanidine, tolazoline, tolbutamide, tolcapone, tolnaftate, tolperisone, tolterodine, topiramate, topotecan, torasemide, tramadol, tramazoline, trandolapril, tranylcypromine, trapidil, trastuzumab, travoprost, trazodone, trepostinil, triamcinolone and triamcinolone derivatives, triamterene, trifluperidol, trifluridine, trimetazidines, trimethoprim, trimipramine, tripelennamine, triprolidine, trifosfamide, tromantadine, trometamol, tropalpine, trovafloxacin, troxerutin, tulobuterol, trypsins, tyramine, tyrothricin, urapidil, ursodeoxycholic acid, theophylline ursodeoxycholic acid, valaciclovir, valdecoxib, valganciclovir, valproic acid, valsartan, vancomycin, vardenafil, vecuronium chloride, venlafaxine, verapamil, verteporfin, vidarabine, vigabatrine, viloxazine, vinblastine, vincamine, vincristine, vindesine, vinorelbine, vinpocetine, viquidil, vitamin D and derivatives of vitamin D, voriconazole, warfarin, xantinol nicotinate, ximelagatran, xipamide, zafirlukast, zalcitabine, zaleplon, zanamivir, zidovudine, ziprasidone, zoledronic acid, zolmitriptan, zolpidem, zoplicone, zotepine and the like.

The active ingredients can if desired also be used in the form of their pharmaceutically acceptable salts or derivatives, and in the case of chiral active ingredients it is possible to employ both optically active isomers and racemates or mixtures of diastereomers. If desired, the compositions of the invention may also comprise two or more active pharmaceutical ingredients.

The Outer Controlling Layer d)

The outer controlling layer d) comprises at least 60, preferably at least 80, particularly preferably 90 to 100, % by weight of one or a mixture of a plurality of (meth)acrylate copolymers composed of 98 to 85 C₁ to C₄ alkyl esters of (meth)acrylic acid and 2 to 15% by weight of methacrylate monomers with a quaternary amino group in the alkyl radical, and, where appropriate, up to 40, preferably up to 20, in particular 0 to 10, % by weight of further pharmaceutically usable polymers. However, is particularly preferred for no further pharmaceutically usable polymers to be present. The data on the % by weight of the abovementioned polymers in the outer controlling layer d) are moreover calculated without taking account of any pharmaceutically usual excipients which are additionally present.

It was one object of the present invention to develop a multiparticulate pharmaceutical form which releases at least 50% of an active pharmaceutical ingredient in less than 8 hours. In order to achieve this object it was found that the outer controlling layer d) has to be comparatively thin. The layer thickness has to be in range of 20 to less than 55, in particular 25 to 50, particularly preferably 30 to 45 μm. The layer thickness can be determined for instance by scanning electron microscopy (SEM) of the pellet structure.

The outer controlling layer d) contains 0,1 to 10, preferred 1 to 6% by weight of glycerol monostearate. The content of 0,1 to 10% by weight of glycerol monostearate is important for providing the comparatively low thickness of the outer controlling layer d) from 20 to less than 55 μm and sufficient stability during the compression process. It was surprisingly found that when other parting agents, such as talc, are used in the outer controlling layer d) in this range of thickness the coatings become leaky or partially damaged during the compression process of the pellets with the outer phase ingredients. By comparing the active ingredient release profiles of pellets that have been compressed with ones which have not been compressed, damaged or leaky coatings can be detected. If the pellets have not become leaky during compression the release profiles are almost the same or identical. If the pellets have become leaky their release profiles are more than 15% faster than those of the non-compressed pellets. With damaged or leaky coatings of the pellets no more controlled release can be expected by the resulting multiparticulate pharmaceutical form.

Glycerol Monostearate

Often the chemical composition of glycerol monostearate products on the market does not exactly correspond to the chemical name indicated. So glycerol monostearate products may contain at least 40, 50, 75, 90, 95 or 99 or even 99,9% by weight of pure glycerol monostearate but may also contain more or less of mono- or diglycerides or fatty acids as well as glycerine or free fatty acids and the like. Suitable glycerol monostearate products may have a hydrophile-lipophile balance (HLB) for instance in the range of 3.5 to 3.8. However the claimed content of glycerol monostearate refers to pure glycerol monostearate present and detectable in the outer controlling layer d) in the pellets of the multparticulate pharmaceutical form for instance by gas phase chromatography (GPC) or NMR or other suitable analytical methods.

The hydrophile-lipophile balance (HLB) is a measure, introduced by Griffin in 1950, of the hydrophilicity and lipophilicity, respectively of non-ionic surfactants. It can be determined experimentally by the titration method after Marszall [See Parfumerie Kosmetik, vol. 60, 1979, pp. 1979, for additional bibliography see for instance Römpps Chemie-Lexikon, 8^{th} ed., vol. 3 (1983).

Appropriate (meth)acrylate copolymers are disclosed for example in EP-A 181 515 or DE patent 1 617 751. They are polymers which are soluble or swellable irrespective of the pH and are suitable for medicament coatings. A possible production process to be mentioned is bulk polymerization in the presence of an initiator which forms free radicals and is dissolved in the monomer mixture. The polymer can likewise be produced by means of solution or precipitation polymerization. The polymer can be obtained in this way in the form of a fine powder, achievable in the case of bulk polymerization by grinding and in the case of solution and precipitation polymerization for example by spray drying.

The (meth)acrylate copolymer is composed of 85 to 98% by weight of free-radical polymerized C₁ to C₄ alkyl esters of acrylic or methacrylic acid and 15 to 2% by weight of (meth)acrylate monomers with a quaternary amino group in the alkyl radical.

Preferred C₁ to C₄ alkyl esters of acrylic or methacrylic acid are methyl acrylate, ethyl acrylate, butyl acrylate, butyl methacrylate and methyl methacrylate.

The particularly preferred (meth)acrylate monomer with quaternary amino groups is 2-trimethylammoniumethyl methacrylate chloride.

An appropriate copolymer may be composed for example of 50-70% by weight of methyl methacrylate, 20-40% by weight of ethyl acrylate and 7-2% by weight of 2-trimethylammoniumethyl methacrylate chloride.

A specifically suitable copolymer comprises 65% by weight of methyl methacrylate, 30% by weight of ethyl acrylate and 5% by weight of 2-trimethylammoniumethyl methacrylate chloride be composed (EUDRAGIT® RS).

A further suitable (meth)acrylate copolymer may be composed for example of 85 to less than 93% by weight of C₁ to C₄ alkyl esters of acrylic or methacrylic acid and more than 7 to 15% by weight of (meth)acrylate monomers with a quaternary amino group in the alkyl radical. Such (meth)acrylate monomers are commercially available and have long been used for release-slowing coatings.

A specifically suitable copolymer comprises for example 60% by weight of methyl methacrylate, 30% by weight of ethyl acrylate and 10% by weight of 2-trimethylammoniumethyl methacrylate chloride (EUDRAGIT® RL).

It is possible where appropriate for up to 40, preferably up to 20, in particular 0 to 10, % by weight of further pharmaceutically usable polymers to be present in the outer controlling layer d). Examples of suitable polymers are:

copolymers of methyl methacrylate and/or ethyl acrylate and methacrylic acid, copolymers of methyl methacrylate, methyl acrylate and methacrylic acid, copolymers of methyl methacrylate, butyl methacrylate and dimethylethyl methacrylate, copolymers of methyl methacrylate, ethyl acrylate and trimethylammoniumethyl methacrylate, copolymers of methyl methacrylate and ethyl acrylate, copolymers of ethyl acrylate, methyl acrylate, butyl methacrylate and methacrylic acid,

polyvinylpyrolidones (PVPs), polyvinyl alcohols, polyvinyl alcohol-polyethylene glycol graft copolymer (Kollicoat®), starch and derivatives thereof, polyvinyl acetate phthalate (PVAP, Coateric®), polyvinyl acetate (PVAc, Kollicoat), vinyl acetate/vinylpyrolidone copolymer (Kollidone® VA64), vinyl acetate: crotonic acid 9:1 copolymer (VAC: CRA, Kollicoat® VAC), polyethylene glycols with a molecular weight above 1000 (g/mol), chitosan, a (meth)acrylate copolymer consisting of 20-40% by weight of methyl methacrylate and 60 to 80% by weight of methacrylic acid, a crosslinked and/or uncrosslinked polyacrylic acid, an Na alginate, and/or a pectin,

celluloses such as, for example, anionic carboxymethyl-cellulose and salts thereof (CMC, Na-CMC, Ca-CMC, Blanose, Tylopur), carboxymethylethylcellulose (CMEC, Duodcell®), hydroxyethylcellulose (HEC, Klucel), hydroxypropylcellulose (HPC), hydroxypropylmethylcellulose (HPMC, Pharmacoat, Methocel, Sepifilm, Viscontran, Opadry), hydroxymethylethylcellulose (HEMC), ethylcellulose (EC, Ethocel®, Aquacoat®, Surelease®), methylcellulose (MC, Viscontran, Tylopur, Methocel), cellulose esters, cellulose glycolate, cellulose acetate phthalate (CAP, Cellulosi acetas, PhEur, cellulose acetate phthalate, NF, Aquateric®), cellulose acetate succinate (CAS), cellulose acetate trimeliate (CAT), hydroxypropylmethylcellulose phthalate (HPMCP, HP50, HP55), hydroxypropylmethylcellulose acetate succinate (HPMCAS-LF, -MF, -HF).

Layer Thicknesses and Proportions by Weight

Optional Core a)

If neutral cores (nonpareilles) are used as carriers, they may be in the range of an average diameter of about 50 to 1500 μm.

Inner Controlling Layer b)

The inner controlling layer comprises
a) a substance having a modulating effect,
b) pharmaceutically usable polymers, waxes, resins and/or proteins,
c) optionally an active ingredient
b) can amount in relation to a) to 50 to 400, preferably 10 to 200, % by weight.
c) can be present in relation to a) and b) in amounts of 10 to 100% by weight.

Active Ingredient Layer c)

The active ingredient layer c) may account for 10 to 400, preferably 50 to 200, % by weight based on the core layer a) and the inner controlling layer b).

Outer Controlling Layer d)

It was one object of the present invention to develop a multiparticulate pharmaceutical form which releases at least 50% of an active pharmaceutical ingredient in less than 8 hours. In order to achieve this object it was found that the outer controlling layer d) has to be comparatively thin. The-layer thickness has to be in range of 20 to less than 55, in particular 25 to 50, particularly preferably 30 to 45 μm. The layer thickness can be determined for instance by scanning electron microscopy (SEM) of the pellet structure.

The outer controlling layer d) may have a proportion by weight of from 2.5 to 100, preferably 10 to 70, particularly preferably 20 to 50, % by weight based on the core layer a), the inner controlling layer b) and the active ingredient layer c).

Excipients Customary in Pharmacy

Layers a), b), c) and d) may additionally and in a manner known per se comprise excipients customary in pharmacy.

Excipients customary in pharmacy, occasionally also referred to as customary additives, are added to the formulation of the invention, preferably during production of the granules or powders. It is, of course, always necessary for all the substances employed to be toxicologically acceptable and usable in particular in medicaments without a risk for patients.

The amounts employed and the use of excipients customary in pharmacy for medicament coatings or layerings are familiar to the skilled worker. Examples of possible excipients or additives customary in pharmacy are release agents, pigments, stabilizers, antioxidants, pore formers, penetration promoters, gloss agents, aromatizing substances or flavourings. They serve as processing aids and are intended to ensure a reliable and reproducible production process and good long-term storage stability or they achieve additional advantageous properties in the pharmaceutical form. They are added to the polymer preparations before processing and may influence the permeability of the coatings, it being possible to utilize this where appropriate as additional control parameter.

Release Agents:

Release agents usually have lipophilic properties and are usually added to the spray suspensions. They prevent agglomeration of the cores during the film coating. Talc, Mg stearate or Ca stearate, ground silica, kaolin or nonionic emulsifiers with an HLB of between 3 and 8 are preferably employed. The usual amounts employed of release agent are between 0.5 to 100% by weight based on the weight of the cores.

Pigments:

Pigments incompatible with the coating agent are in particular those pigments which, if added directly to the (meth)acrylate copolymer dispersion, e.g. by stirring in, in the usual amounts used of, for example, 20 to 400% by weight based on the dry weight of the (meth)acrylate copolymer, lead to destabilization of the dispersion, coagulation, to signs of inhomogeneity or similarly unwanted effects. The pigments to be used are moreover of course non-toxic and suitable for pharmaceutical purposes. Concerning this, see also, for example: Deutsche Forschungsgemeinschaft, Farbstoffe fur Lebensmittel, Harald, Boldt Verlag K G, Boppard (1978); Deutsche Lebensmittelrundschau 74, No. 4, p. 156 (1978); Arzneimittelfarbstoffverordnung AmFarbV of 25.08.1980.

Pigments incompatible with the coating agent may be for example alumina pigments. Examples of incompatible pigments are orange yellow, cochineal red lake, coloured pigments based on alumina or azo dyes, sulphonic acid dyes, orange yellow S (E110, C.I. 15985, FD&C Yellow 6), indigo carmine (E132, C.I. 73015, FD&C Blue 2), tartrazine (E 102, C.I. 19140, FD&C Yellow 5), Ponceau 4R (E 125, C.I. 16255, FD&C Cochineal Red A), quinoline yellow (E 104, C.I. 47005, FD&C Yellow 10), erythrosine (E127, C.I. 45430, FD&C Red 3), azorubine (E 122, C.I. 14720, FD&C Carmoisine), amaranth (E 123, C.I. 16185, FD&C Red 2), acid brilliant green (E 142, C.I. 44090, FD&C Green S).

The E numbers indicated for the pigments relate to an EU numbering. Concerning this, see also “Deutsche Forschungsgemeinschaft, Farbstoffe für Lebensmittel, Harald Boldt Verlag K G, Boppard (1978); Deutsche Lebensmittelrundschau 74, No. 4, p. 156 (1978); Arzneimittelfarbstoffverordnung AmFarbV of 25.08.1980. The FD&C numbers relate to the approval in food, drugs and cosmetics by the U.S. food and drug administration (FDA) described in: U.S. Food and Drug Administration, Center for Food Safety and Applied Nutrition, Office of Cosmetics and Colors: Code of Federal Regulations—Title 21 Color Additive Regulations Part 82, Listing of Certified Provisionally Listed Colors and Specifications (CFR 21 Part 82).

Plasticizers

Further additives may also be plasticizers. The usual amounts are between 0 and 50, preferably 5 to 20, % by weight based for example on the (meth)acrylate copolymer of the outer layer d).

Plasticizers may influence the functionality of the polymer layer, depending on the type (lipophilic or hydrophilic) and added amount. Plasticizers achieve through physical interaction with the polymers a reduction in the glass transition temperature and promote film formation, depending on the added amount. Suitable substances usually have a molecular weight of between 100 and 20 000 and comprise one or more hydrophilic groups in the molecule, e.g. hydroxyl, ester or amino groups.

Examples of suitable plasticizers are alkyl citrates, glycerol esters, alkyl phthalates, alkyl sebacates, sucrose esters, sorbitan esters, diethyl sebacate, dibutyl sebacate and polyethylene glycols 200 to 12 000. Preferred plasticizers are triethyl citrate (TEC), acetyl triethyl citrate (ATEC) and dibutyl sebacate (DBS). Mention should additionally be made of esters which are usually liquid at room temperature, such as citrates, phthalates, sebacates or castor oil. Esters of citric acid and sebacic acid are preferably used.

Addition of the plasticizers to the formulation can be carried out in a known manner, directly, in aqueous solution or after thermal pretreatment of the mixture. It is also possible to employ mixtures of plasticizers.

Processes for Producing a Multilayer Pharmaceutical Form (Pellets)

The pellets (multilayer pharmaceutical form) can be produced in a manner known per se by means of usual pharmaceutical processes such as direct compression, compression of dry, wet or sintered granules, extrusion and subsequent rounding off, wet or dry granulation or direct pelleting (e.g. on plates) or by binding of powders (powder layering) onto active ingredient-free beads or cores (nonpareilles) or active ingredient-containing particles, by means of spray processes or fluidized bed granulation. Application of the outer controlling layer d) can take place by means of known and usual processes such as, for example, spray application of polymer solutions or polymer dispersions.

The Multiparticultate Pharmaceutical Form

The multiparticulate pharmaceutical form contains 20 to 60, preferred 40 to 55% by weight of the multilayered pellets. The multilayered pellets are compressed in mixture with 80 to 40%, preferred 60 to 45% by weight of an outer phase which consists from 50 to 100, preferred from 70 to 90% by weight of a cellulose or a derivate of cellulose. Cellulose or an or derivates of cellulose have the advantage of high compressability. So this respectively these ingredients contribute to achieve an multiparticulate pharmaceutical form by compression of the pellets in mixture with the outer phase without causing damage to the coatings of the pellets. Compression may be carried out with a pressure of 5 to 40, respectively 10 to 20 kN.

Cellulose shall mean cellulose consisting essentially of linear cellulose molecules without branches for instance microcrystalline cellulose with the exception of crosslinked celluloses.

Derivates of cellulose shall mean derivates of cellulose consisting essentially of linear cellulose molecules without branches for instance hydroxyl propyl cellulose, ethyl cellulose, propyl cellulose, methylcellulose, hydroxyl ethyl cellulose or cellactose with the exception of crosslinked celluloses.

Beside the cellulose or derivates of cellulose optionally further pharmaceutical excipients may be present in the outer phase in amounts of 0 to 50, preferred 20 to 40% by weight. Further pharmaceutical excipients in the outer phase may be without limiting the invention for instance branched or crosslinked celluloses functioning as disintegrants, talc as a gliding agent to support the compression process and the like.

It was one object of the present invention to develop a multiparticulate pharmaceutical form which releases at least 50% of an active pharmaceutical ingredient in less than 8 hours. In order to achieve this object it was found that the outer controlling layer d) has to be comparatively thin. The layer thickness has to be in range of 20 to less than 55, in particular 25 to 50, particularly preferably 30 to 45 μm. The layer thickness can be determined for instance by electron microscopy of the pellet structure.

Process for Producing a Multiparticulate Pharmaceutical Form

A multiparticulate pharmaceutical form according to the invention may be produced by first producing pellets with the multilayer structure in a manner known per se by means of pharmaceutically customary processes such as by direct compression, compression of dry, wet or sintered granules, extrusion and subsequent rounding off, wet or dry granulation or direct pelleting or by binding of powders (powder layering) onto active ingredient-free beads or neutral cores (nonpareilles) or active ingredient-containing particles or by means of spraying processes or fluidized bed granulation and secondly producing the multiparticulate pharmaceutical form by compression of 10 to 60% by weight of the pellets with the multilayer structure in mixture with 90 to 40% by weight of an outer phase which consists from 50 to 100% by weight or more of a cellulose or a derivate of cellulose and optionally 0 to 50% by weight of further pharmaceutical excipients.

The Compression process may be carried out on single punch presses or rotary presses with punches of different shape and a pressure of 5 to 40, respectively 10 to 20 kN.

Additional Outer Polymer Film Coating

The multiparticulate pharmaceutical form may carry an additional outer polymer film coating which may function as a carrier for pigments, as a moisture barrier, for taste masking or providing resistance against the influence of gastric juices. Examples for polymers for such an outer coating are hydroxypropyl cellulose as a carrier for pigments or (meth)acrylic polymer containing residues of dimethylaminoethylmethacrylat monomers (EUDRAGIT® E type polymers) as moisture barrier and/or taste masking and (meth)acrylic polymers containing (meth)acrylic acid residues (EUDRAGIT® L, S, L100-55 or ES type polymers) for resistance against the influence of gastric juices.

Possible Release Characteristics

The multilayer pharmaceutical form is particularly suitable for achieving specific active ingredient release characteristics. Mention should be made of active ingredient release characteristics of zero order (linear), 1st order (accelerated), fast-slow, slow-fast release characteristics.

Dosage Forms/Uses

The multilayer pharmaceutical forms of the invention are initially in the form of tablets or pellets. These can in turn be used as ingredient of a multiparticulate pharmaceutical form, of pellet-containing tablets, minitablets, capsules, sachets, effervescent tablets or powders for reconstitution. It is possible according to the invention for multiparticulate pharmaceutical forms also to include in particular mixtures of formulated pellets comprising different active ingredients. A further possibility is for multiparticulate pharmaceutical forms of the invention to comprise pellet populations which are loaded with one and the same active ingredient but are differently formulated and show different release profiles. It is possible in this way for mixed release profiles of one or more active ingredients to be achieved and for a more refined adaptation for the desired therapy to be carried out via the mixtures.

### EXAMPLES

EUDRAGIT® RS=copolymer of 65% by weight of methyl methacrylate, 30% by weight of ethyl acrylate and 5% by weight of 2-trimethylammoniumethyl methacrylate chloride, 30% dispersion; EUDRAGIT® RS 30D 30% dispersion;

EUDRAGIT® NE 30D=copolymer of 50% by weight of methyl methacrylate and 50% by weight of ethyl acrylate.

Preparation of Pellets (Layers a-c)

1000 g of sodium chloride are granulated in a compulsory mixer with 300 g of EUDRAGIT® NE 30 D (equivalent to 100 g of copolymer)

A mixture of 1290 g of theophylline powder, 65 g of Kollidon 25 and 6.5 g of Aerosil 200 are sprinkled onto 700 g of the cores produced in this way with slow-release modulator delivery in a coating pan and bound to the core material by simultaneous spraying of a solution of 33 g of theophylline and 10 of Kollidon 25 in 500 g of demineralized water.

Application of an Outer Controlling Layer d) Consisting of a Release-Slowing Coating with (EUDRAGIT® RS)

The active ingredient-coated pellets with layers a, b and c are coated with EUDRAGIT® RS 30 D (layer d) in a fluidized bed apparatus (GLATT 3.1, top spray), applying various amounts of polymer providing coatings of different thicknesses (from 20-80 μm), investigated by SEM.

Two formulations are applied:

Preparation A (Talc)

Aqueous Coating suspension formulation comprising in dispersion: 8.5% by weight solid polymer, 4,2% by weight talc, 1.7% by weight triethyl citrate.

The Coating suspension is prepared by dispersing triethyl citrate and talc in water separately and pouring it into EUDRAGIT® RS 30 D and gently stirring. Stirring is continued during storage and spraying.

Preparation B (Glycerol Monostearate)

Aqueous Coating suspension formulation comprising in dispersion: 8.5% by weight solid polymer, 0,21% 0.425% by weight glycerol monostearate (=0.425% IMVITORTTM 900, containing approximately 45% glycerol monostearate), − and 1.7% by weight triethyl citrate The coating suspension is prepared by dispersing triethyl citrate and glycerol monostearate in heated water of 65° C.-70° C., cooling the emulsion to room temperature, pouring it into EUDRAGIT® RS 30 D and gently stirring. Stirring is continued during storage and spraying

Approximated process parameters

| | |
|---|---|
| Inlet air temperature: | 30-40° C. |
| Product temperature: | 24-27° C. |
| Outlet air temperature | 24-30° C. |
| Spray rate: | 10 g/kg * min) |
| Drying process: | 60 min fluidization at 40° C. |
| | and 24 h in a convection oven |
| | at 40° C. |

Preparation of Disintegrating Multiparticulate Form (Tablets):

1 kg of a mixture comprising 50% by weight coated pellets including the outer coating d), 43.5% by weight microcrystalline cellulose (Vivapur™ 102), 5% by weight Ac-Di-Sol, 0.5% by weight AEROSIL™ 200, 2% by weight talc and 0.5% by weight magnesium stearate is prepared by blending the ingredients (except magnesium stearate) for 20 min, adding magnesium stearate and blending for another 1 min.

The mixture is compressed on a rotary press using 2 oblong punches (9×12 mm, standard concave)at 16 rpm. Tablets of 415 mg-450 are obtained with a hardness of more than 100 N and a friability of less than 1%.

Dissolution Methodology

Dissolution studies were performed the basket apparatus (USP Type I) at 100 rpm, using EP phosphate buffer 6.8 (European Pharmacopoeia) as test medium. Samples were taken after different periods and the dissolved theophylline detected by UV spectrophotometer at maximum of extinction.

### Example I

### Not According to the Invention

Pellets are prepared as described above applying an outer coating preparation 4 A, being 75-80 μm thick. Multiparticulate form (tablets) are prepared as described above.

The dissolution plot of the pellets show a zero order profile, i.e. it is virtually linear. The quantity of drug released after 8 hours is less than 50%. The dissolution profile of the tablets do not differ from the dissolution profile of the pellets more than 15% by weight.

### Example II

### Not According to the Invention

Pellets are prepared as described above, applying an outer coating preparation 4 A, being 55-60 μm thick. Multiparticulate form (tablets) are prepared as described above.

The dissolution plot of the pellets show a zero order profile, i.e. it is virtually linear. The quantity of drug released after 8 hours is less than 50%. The dissolution profile of the tablets do not differ from the dissolution profile of the pellets more than 15% by weight.

### Example III

### Not According to the Invention

Pellets are prepared as described above applying an outer coating preparation 4 A, being 30-35 μm thick. Multiparticulate form (tablets) are prepared as described above.

The dissolution plot of the pellets show a zero order profile, i.e. it is virtually linear. The quantitiy of drug released after 8 hours is more than 50%. The dissolution profile of the tablets differ from the dissolution profile of the pellets more than 15% by weight.

### Example IV

### According to the Invention

Pellets are prepared as described above applying an outer coating preparation 4 B, being 20-25 μm thick. Multiparticulate form (tablets) are prepared as described above.

The dissolution plot of the pellets show a zero order profile, i.e. it is virtually linear. The quantity of drug released after 8 hours is more than 50%. The dissolution profile of the tablets do not differ from the dissolution profile of the pellets more than 15% by weight.

### Example V

### According to the Invention

Pellets are prepared as described above applying an outer coating preparation 4 B, being 30-35 μm thick. Multiparticulate form (tablets) are prepared as described above.

The dissolution plot of the pellets show a zero order profile, i.e. it is virtually linear. The quantity of drug released after 8 hours is more than 50%. The dissolution profile of the tablets do not differ from the dissolution profile of the pellets more than 15% by weight.

### Example VI

### According to the Invention

Pellets are prepared as described above applying an outer coating preparation 4 B, being 45-50 μm thick. Multiparticulate form (tablets) are prepared as described above.

The dissolution plot of the pellets show a zero order profile, i.e. it is virtually linear. The quantity of drug released after 8 hours is more than 50%. The dissolution profile of the tablets do not differ from the dissolution profile of the pellets more than 15% by weight.

1. A multiparticulate pharmaceutical form, comprising pellets with a multilayer structure for controlled active ingredient release, comprising
a) optionally a neutral core (nonpareilles),
b) an inner controlling layer comprising a substance having a modulating effect, which is embedded in a matrix which influences the delivery of the modulatory substance and which comprises pharmaceutically usable polymers, waxes, resins and/or proteins, and where appropriate an active ingredient,
c) an active ingredient layer comprising an active pharmaceutical ingredient and, where appropriate, a substance having a modulating effect,
d) an outer controlling layer comprising at least 60% by weight of one or a mixture of a plurality of (meth)acrylate copolymers of comprising from 98 to 85 C₁ to C₄ alkyl esters of (meth)acrylic acid and from 2 to 15% by weight of methacrylate monomers with a quaternary amino group in the alkyl radical, and, where appropriate, up to 40% by weight of further pharmaceutically usable polymers, where the layers may additionally comprise pharmaceutically usual excipients, where the outer controlling layer has a thickness from 20 to less than 55 μm and comprises from 0.1 to 10% by weight of glycerol monostearate, where the multiparticulate pharmaceutical form comprises from 20 to 60% by weight of the pellets, which are compressed in a mixture comprising from 80 to 40% by weight of an outer phase which consists of from 50 to 100% by weight of a cellulose or a derivate of cellulose and optionally from 0 to 50% by weight of further pharmaceutical excipients.

2. The multiparticulate pharmaceutical form according to claim 1, wherein the matrix of the inner controlling layer comprises one or more of the following polymers:
copolymers of methyl methacrylate and/or ethyl acrylate and methacrylic acid, copolymers of methyl methacrylate, methyl acrylate and methacrylic acid, copolymers of methyl methacrylate, butyl methacrylate and dimethylethyl methacrylate, copolymers of methyl methacrylate, ethyl acrylate and trimethylammoniumethyl methacrylate, copolymers of methyl methacrylate and ethyl acrylate, copolymers of ethyl acrylate, methyl acrylate, butyl methacrylate and methacrylic acid,
polyvinylpyrolidones (PVPs), polyvinyl alcohols, polyvinyl alcohol-polyethylene glycol graft copolymer, starch and derivatives thereof, polyvinyl acetate phthalate (PVAP), polyvinyl acetate (PVAc), vinyl acetate/vinylpyrolidone copolymer, vinyl acetate: crotonic acid 9:1 copolymer (VAC: CRA), polyethylene glycols with a molecular weight above 1000 (g/mol), chitosan, a (meth)acrylate copolymer consisting of 20-40% by weight of methyl methacrylate and 60 to 80% by weight of methacrylic acid, a crosslinked and/or uncrosslinked polyacrylic acid, an Na alginate, and/or a pectin, and/or
celluloses including anionic carboxymethylcellulose and salts thereof (CMC, Na-CMC, Ca-CMC), carboxymethylethylcellulose (CMEC), hydroxyethylcellulose (HEC), hydroxypropylcellulose (HPC), hydroxypropylmethylcellulose (HPMC), hydroxymethylethylcellulose (HEMC), ethylcellulose (EC), methylcellulose (MC), cellulose esters, cellulose glycolate, cellulose acetate phthalate (CAP), cellulose acetate succinate (CAS), cellulose acetate trimeliate (CAT), hydroxypropylmethylcellulose phthalate (HPMCP), hydroxypropylmethylcellulose acetate succinate (HPMCAS).

3. The multiparticulate pharmaceutical form according to claim 1, wherein the matrix of the inner controlling layer comprises a wax, selected from one or more of carnauba wax and/or beeswax.

4. The multiparticulate pharmaceutical form according to claim 1, wherein the matrix of the inner controlling layer comprises resin shellac.

5. The multiparticulate pharmaceutical form according to claim 1, wherein the matrix of the inner controlling layer comprises a protein, selected from one or more of albumin, gelatin, zein, collagen, gluten and/or a lectin.

6. The multiparticulate pharmaceutical form according to claim 1, wherein the substance having a modulating effect has a molecular weight below 500 and is in solid form and is ionogenic.

7. The multiparticulate pharmaceutical form according to claim wherein the substance having a modulating effect is soluble in water.

8. The multiparticulate pharmaceutical form according to claim 6, wherein the substance having a modulating effect is selected from one or more of an organic acid, a salt of an organic acid, and/or a salt of an inorganic acid.

9. The multiparticulate pharmaceutical form according to claim 6, wherein the substance having a modulating effect is selected from one or more of succinic acid, citric acid, fumaric acid, malic acid, maleinic acid tartaric acid, laurylsulphuric acid, a salt of these acids or a salt of the following anions: taurochlolate and other cholates, chlorides, acetates, lactates, phosphates and/or sulphates.

10. The multiparticulate pharmaceutical form according to claim 1, wherein the multiparticulate pharmaceutical form further comprises an additional outer polymer film coating selected from one or more of a carrier for pigments, as a moisture barrier, a taste masking coating and/or a gastric juice resistant coating.

11. A process for producing the multiparticulate pharmaceutical form according to claim 1, wherein said process comprises:
producing pellets with the multilayer structure.
